(19) United States
(12) Patent Application Publication
(10) Pub. No.: US 2025/0205060 A1
(43) Pub. Date: Jun. 26, 2025

(54) ARTICULATING INTERBODY FUSION DEVICES AND METHODS OF MANUFACTURE

(71) Applicant: HT MEDICAL, LLC, Tulsa, OK (US)

(72) Inventors: Ryan PHILLIPS, Orlando, FL (US); Nicholas M. CORDARO, San Diego, CA (US); Matthew COLMAN, Chicago, IL (US); Joshua MILLER, Knoxville, TN (US)

(21) Appl. No.: 18/991,985

(22) Filed: Dec. 23, 2024

Related U.S. Application Data
(60) Provisional application No. 63/614,547, filed on Dec. 23, 2023.

(51) Int. Cl. A61F 2/44

(52) U.S. Cl. CPC A61F 2/4465; A61F 2220/0025

(57) ABSTRACT

An articulating interbody fusion device may include a body including a plurality of interconnected pores, and a recess portion having a first border portion and a second border portion spaced apart from the first border portion. The articulating interbody fusion device may further include an articulating connector configured to be positioned within the recess portion. One of the body and the articulating connector may include an aperture, the other of the body and the articulating connector may include a post positioned within the aperture, the articulating connector may be rotatable relative to the body, and the articulating connector may be irremovable from the body.

### CROSS-REFERENCE TO RELATED APPLICATIONS

The present disclosure claims the benefit of U.S. Provisional Patent Application Ser. No. 63/614,547, filed on Dec. 23, 2023 and entitled ARTICULATING INTERBODY FUSION DEVICE, which is incorporated by reference as though set forth herein in its entirety.

### TECHNICAL FIELD

The present disclosure relates to systems and methods for use in orthopedic surgery. More specifically, the present disclosure relates to articulating interbody fusion systems and surgical methods.

### BACKGROUND

Interbody fusion devices are advanced implants used in spinal surgery to treat conditions such as degenerative disc disease, spinal instability, and spondylolisthesis. Unlike traditional interbody fusion implants that are rigid, articulating devices are designed to allow for more control over the location and position of the implant, increasing accuracy in the process of placing the device in the spine.

Articulating interbody fusion devices are crucial for stabilizing the spine and alleviating pain in patients with various spinal conditions, however, traditional manufacturing methods for these devices often involve complex machining processes, which can be costly, time-consuming, and limit design flexibility. As a result, there is a growing need for alternative manufacturing methods which improve the manufacturing process of articulating interbody fusion devices. By applying modern manufacturing techniques in a novel manner, the manufacturing process of articulating interbody fusion devices can be simplified, reducing both the cost and time needed in the process, while simultaneously increasing flexibility in the design of the device.

### SUMMARY

The various systems and methods of the present disclosure have been developed in response to the present state of the art, and in particular, in response to the problems and needs in the art that have not yet been fully solved by currently available articulating interbody fusion devices and methods of manufacture.

In some embodiments, an articulating interbody fusion device may include a body including a plurality of interconnected pores, and a recess portion having a first border portion and a second border portion spaced apart from the first border portion. The articulating interbody fusion device may further include an articulating connector configured to be positioned within the recess portion. One of the body and the articulating connector may include an aperture, the other of the body and the articulating connector may include a post positioned within the aperture, the articulating connector may be rotatable.

In the articulating interbody fusion device of any preceding paragraph, the post may be monolithically formed with the body.

In the articulating interbody fusion device of any preceding paragraph, the post may extend from the first border portion to the second border portion.

In the articulating interbody fusion device of any preceding paragraph, the articulating connector may include the aperture and an outer perimeter, wherein the aperture may be entirely encircled by the outer perimeter.

In the articulating interbody fusion device of any preceding paragraph, wherein the first border portion and the second border portion may lack interconnected pores.

In the articulating interbody fusion device of any preceding paragraph, the plurality of interconnected pores may provide continuous pathways from a first side of the body to an opposing second side of the body.

In the articulating interbody fusion device of any preceding paragraph, the body may include a modulus of elasticity similar to that of a bone.

In the articulating interbody fusion device of any preceding paragraph, the device may further include a printing orientation, wherein the articulating interbody fusion device may include one or more overhang surfaces that are not perpendicular to the printing orientation.

In the articulating interbody fusion device of any preceding paragraph, the articulating connector may be formed as a single monolithic piece.

In the articulating interbody fusion device of any preceding paragraph, the device may further include a first stop surface configured to limit rotational movement of the articulating connector at a first position and a second stop surface configured to limit rotational movement of the articulating connector at a second position, wherein an angle between the first position and the second position may be in a range of 50° to 70°.

In some embodiments, a method of manufacturing an articulating interbody fusion device may include additively manufacturing a body and additively manufacturing an articulating connector such that the articulating connector is positioned to rotatably engage the body. Additively manufacturing the body and the articulating connector may include additively manufacturing the body and the articulating connector together, with the body and the articulating connector uncoupled to each other by a support material during manufacturing.

In the method of any preceding paragraph, additively manufacturing the body may include forming one or more notches on a distal surface of the body and additively manufacturing the body without utilizing the support material.

In the method of any preceding paragraph, additively manufacturing the articulating connector may include forming a notch on the articulating connector and additively manufacturing the articulating connector without utilizing the support material.

In the method of any preceding paragraph, the body may include a post positioned within an aperture of the articulating connector, and additively manufacturing the body may include additively manufacturing the body with the post and a remainder of the body uncoupled to each other by the support material, aside from a junction between the post and the remainder of a support structure.

In the method of any preceding paragraph, additively manufacturing the body may include forming a notch on the post and manufacturing the post without utilizing the support material.

In the method of any preceding paragraph, additively manufacturing the body and additively manufacturing the articulating connector may include forming one or more angled overhang surfaces that are not perpendicular to a printing orientation so that the articulating interbody fusion device is additively manufactured as an assembly without support structures.

In the method of any preceding paragraph, the articulating connector may be additively manufactured such that the articulating connector is irremovable from the body.

In the method of any preceding paragraph, additively manufacturing the body may include forming a plurality of interconnected pores, forming a recess portion comprising a first border portion lacking the interconnected pores and a second border portion lacking the interconnected pores spaced apart from the first border portion, and forming a post extending from the first border portion to the second border portion. Additively manufacturing the articulating connector may include forming the articulating connector within the recess portion, wherein the articulating connector may include an outer perimeter and an aperture entirely encircled by the outer perimeter. The post may be positioned within the aperture, and the articulating connector may be rotatable relative to the post.

In the method of any preceding paragraph, additively manufacturing the articulating connector may further include forming a first stop surface configured to limit rotational movement of the articulating connector at a first position and forming a second stop surface configured to limit rotational movement of the articulating connector at a second position, wherein an angle between the first position and the second position may be in a range of 50° to 70°.

In the method of any preceding paragraph, additively manufacturing the body and the articulating connector may include depositing layers so that all overhang surfaces are configured with an angle less than a maximum overhang angle.

In some embodiments an articulating interbody fusion device may be formed by additively manufacturing a body and additively manufacturing an articulating connector together with the body such that the articulating connector is positioned to rotatably engage the body. The articulating interbody fusion device may include the body including a plurality of interconnected pores, a recess portion comprising a first border portion lacking the interconnected pores and a second border portion lacking the interconnected pores spaced apart from the first border portion, and a post extending from the first border portion to the second border portion. The articulating interbody fusion device may further include the articulating connector positioned within the recess portion. The articulating connector may include an outer perimeter and an aperture entirely encircled by the outer perimeter, the post may be positioned within the aperture, and the articulating connector may be irremovable from the body.

In the articulating interbody fusion device of any preceding paragraph, the articulating interbody fusion device may include one or more angled overhang surfaces that are not perpendicular to a printing orientation so that the articulating interbody fusion device may be additively manufactured as an assembly without support structures for the angled overhang surfaces.

In the articulating interbody fusion device of any preceding paragraph, additively manufacturing the body and the articulating connector may include additively manufacturing the body and the articulating connector uncoupled to each other by support material during manufacturing.

In the articulating interbody fusion device of any preceding paragraph, additively manufacturing the articulating connector may include forming one or more notches on a distal surface of the articulating connector and additively manufacturing the articulating connector without utilizing a support material.

In the articulating interbody fusion device of any preceding paragraph, additively manufacturing the body and the articulating connector may include depositing layers so that all overhang surfaces are configured with an angle less than a maximum overhang angle.

These and other features and advantages of the present disclosure will become more fully apparent from the following description and appended claims or may be learned by the practice of the implants, systems, and methods set forth hereinafter.

### BRIEF DESCRIPTION OF THE DRAWINGS

Exemplary embodiments of the disclosure will become more fully apparent from the following description and appended claims, taken in conjunction with the accompanying drawings. Understanding that these drawings depict only exemplary embodiments and are, therefore, not to be considered limiting of the scope of the appended claims, the exemplary embodiments of the disclosure will be described with additional specificity and detail through use of the accompanying drawings in which:

FIG. 1A is a perspective view of an articulating interbody fusion device according to an embodiment of the present disclosure.

FIG. 1B is a perspective section view of the articulating interbody fusion device of FIG. 1A.

FIG. 2A is a top view of the articulating interbody fusion device of FIG. 1A.

FIG. 2B is a front view of the articulating interbody fusion device of FIG. 1A.

FIG. 3A is a right-side view of the articulating interbody fusion device of FIG. 1A.

FIG. 3B is a left-side view of the articulating interbody fusion device of FIG. 1A.

FIG. 4A is a perspective view of a body of the articulating interbody fusion device of FIG. 1A.

FIG. 4B is a perspective section view of the body of FIG. 4A.

FIG. 5A is a top view of the body of FIG. 4A.

FIG. 5B is a front view of the body of FIG. 4A.

FIG. 6A is a top section view of the body of FIG. 4A.

FIG. 6B is a right-side view of the body of FIG. 4A.

FIG. 7A is a perspective view of an articulating connector of an articulating interbody fusion device according to an embodiment of the present disclosure.

FIG. 7B is a top view of the articulating connector of FIG. 7A.

FIG. 8A is a right-side view of the articulating connector of FIG. 7A.

FIG. 8B is a left-side view of the articulating connector of FIG. 7A.

FIG. 9A is a front view and a right-side view of the articulating interbody fusion device of FIG. 1A with the articulating connector in an insertion position according to an embodiment of the present disclosure.

FIG. 9B is a top section view of the articulating interbody fusion device of FIG. 9A.

FIG. 10A is a front view and a right-side view of the articulating interbody fusion device of FIG. 1A with the articulating connector in an implanted position according to an embodiment of the present disclosure.

FIG. 10B is a top section view of the articulating interbody fusion device of FIG. 10A.

FIG. 11 is a schematic representation of additive manufacturing material layers.

FIG. 12A is a front view of an articulating interbody fusion device according to an embodiment of the present disclosure.

FIG. 12B is a top view of the articulating interbody fusion device of FIG. 12A.

FIG. 13A is a top section view of an articulating connector according to an embodiment of the present disclosure.

FIG. 13B is a side view of the articulating connector of FIG. 13A.

It is to be understood that the drawings are for purposes of illustrating the concepts of the present disclosure and may not be drawn to scale. Furthermore, the drawings illustrate exemplary embodiments and do not represent limitations to the scope of the present disclosure.

### DETAILED DESCRIPTION

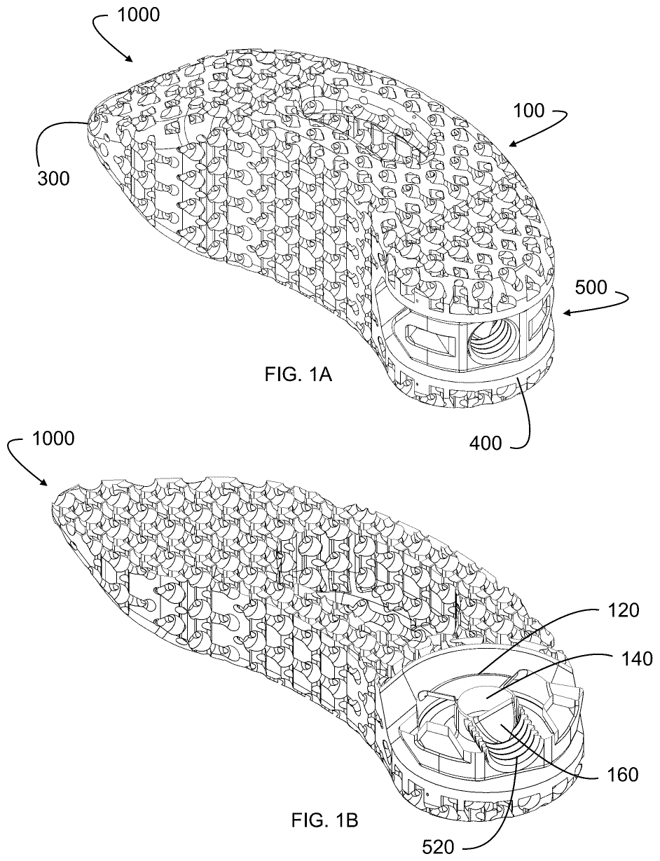

Exemplary embodiments of the disclosure will be best understood by reference to the drawings, wherein like parts are designated by like numerals throughout. It will be readily understood that the components of the disclosure, as generally described and illustrated in the Figures herein, could be arranged and designed in a wide variety of different configurations. Thus, the following more detailed description of the embodiments of the apparatus, system, and method, as represented in FIG. 1A through FIG. 10B, is not intended to limit the scope of the claims, but is merely representative of exemplary embodiments of the present disclosure.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Not every feature of each embodiment is labeled in every figure in which that embodiment appears, in order to keep the figures clear. Similar reference numbers (for example, those that are identical except for the first numeral) may be used to indicate similar features in different embodiments.

Standard medical planes of reference and descriptive terminology are employed in this specification. While these terms are commonly used to refer to the human body, certain terms are applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator.

The present disclosure illustrates devices for a transforaminal lumbar interbody fusion (TLIF) for the purposes of illustrating the concepts of the present design. However, it will be understood that other variations and uses are contemplated including, but not limited to, devices for a posterior lumbar interbody fusion (PLIF), devices for anterior lumbar interbody fusion (ALIF), devices for interbody fusion of the thoracic spine, etc.

The present disclosure is related to U.S. patent application Ser. No. 17/157,322, filed on Jan. 25, 2021 and entitled ADDITIVE MANUFACTURED TITANIUM BONE DEVICE, which is incorporated by reference as though set forth herein in its entirety.

FIG. 1A is a perspective view of an articulating interbody fusion device 1000 according to an embodiment of the present disclosure. FIG. 1B is a perspective section view of the articulating interbody fusion device 1000. In an embodiment, the articulating interbody fusion device 1000 may be configured for deployment within an intervertebral space and may facilitate fusion of two adjacent vertebral bodies. In an embodiment, the spinal fusion may be facilitated via bone in-growth through a porous structure of the articulating interbody fusion device 1000. In an embodiment, the articulating interbody fusion device may be configured as a spacer between two adjacent vertebral bodies.

In an embodiment, the articulating interbody fusion device 1000 may have a body 100 and an articulating connector 500. The articulating interbody fusion device 1000 may have a superior surface configured to engage a superior vertebral body and an inferior surface configured to engage an inferior vertebral body. In an embodiment, the articulating interbody fusion device 1000 may have a proximal end 400 with an articulating connector 500 and a distal end 300.

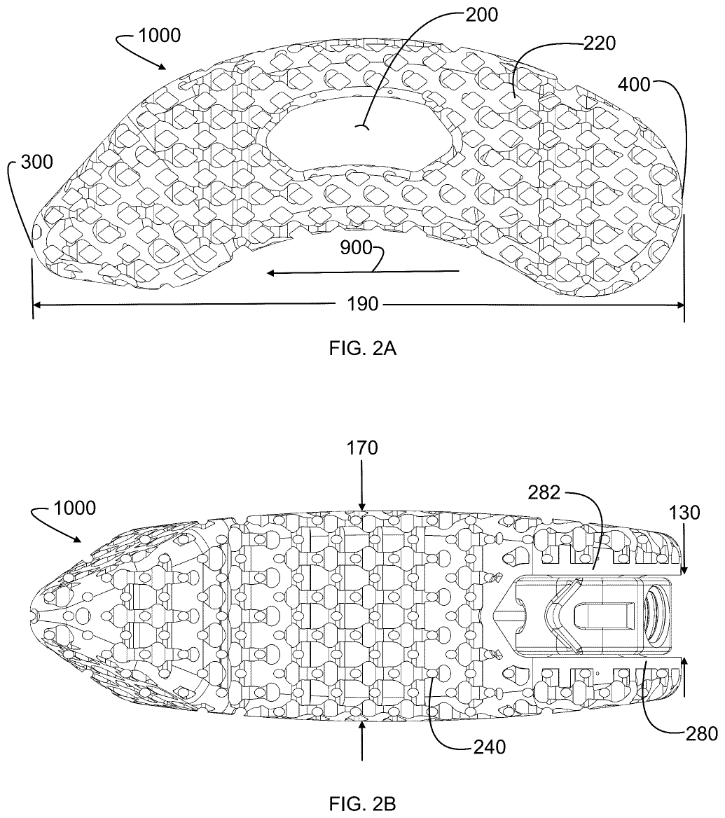

FIG. 2A is a top view of the articulating interbody fusion device 1000 and FIG. 2B is a front view of the articulating interbody fusion device 1000. The top view may represent a superior-inferior plane, i.e., a view along the superior-inferior direction, orthogonal to a plane perpendicular to the superior-inferior direction. The front view may represent an anterior-posterior plane, i.e., a view perpendicular to a superior-inferior direction. In an embodiment, the articulating interbody fusion device 1000 may have a length 190. The length 190 may represent a distance from an proximal end 400 to a distal end 300 as measured in a superior-inferior plane. The articulating interbody fusion device 1000 may be configured such that the length 190 is within a range of lengths from 20 mm to 40 mm. In an embodiment, the articulating interbody fusion device 1000 may be one of a set of differently-sized implants, each having a different length.

In an embodiment, the articulating interbody fusion device 1000 may have a height 170. The height 170 may represent a distance from a bottom end to a top end as measured in an anterior-posterior plane. The articulating interbody fusion device 1000 may be configured such that the height 170 is within a range of heights from 5 mm to 15 mm. In an embodiment, the articulating interbody fusion device 1000 may be one of a set of differently-sized implants, each having a different height.

In an embodiment, the articulating interbody fusion device 1000 may be configured with a lordosis angle. The lordosis angle may be configured so that the height 170 at the proximal end 400 of the articulating interbody fusion device 1000 and the height 170 at the distal end 300 of the articulating interbody fusion device 1000 result are not equal wherein the resulting angle between inferior and superior surfaces is within a range of 1° to 10°. Alternatively, the height 170 at the proximal end 400 of the articulating interbody fusion device 1000 and the height 170 at the distal end 300 of the articulating interbody fusion device 1000 may be generally equal resulting in a 0° lordosis angle.

The desired lordosis angle may depend upon the desired orientation for the articulating interbody fusion device 1000 between the vertebrae. Specifically, if the articulating interbody fusion device 1000 is to be oriented with the length 190 parallel to the medial-lateral direction, a 0° lordosis angle may be preferred. Conversely, if the articulating interbody fusion device 1000 is to be oriented with the length 190 parallel to the anterior-posterior direction, or between the medial-lateral and anterior-posterior directions, a positive lordosis angle, or even a negative lordosis angle, may be desirable.

In an embodiment, the articulating interbody fusion device 1000 may have a central aperture 200. The central aperture 200 may be configured to receive bone graft material or other suitable materials that are known in the art to promote fusion of adjacent vertebral bodies. The central aperture 200 may extend from the superior surface to the inferior surface to permit growth of a column of bone between the superior surface and the inferior surface.

In an embodiment, the articulating interbody fusion device 1000 may have a plurality of top lattice apertures 220. The plurality of top lattice apertures 220 may be arranged in a two-dimensional array throughout the top surface. In an embodiment, the two-dimensional array of top lattice apertures 220 may be configured so the adjacent rows and/or columns may be staggered. Alternatively, the two-dimensional array of top lattice apertures 220 may be configured so the adjacent rows and/or columns may be equally spaced. The plurality of top lattice apertures 220 may extend through the body 100 of the articulating interbody fusion device 1000 providing continuous pathways from the superior surface of the porous structure to the inferior surface of the porous structure. In an embodiment, each of the plurality of top lattice apertures 220 may be configured as shape from one or more of: a diamond; a circle; an oval; a square; a triangle; a pentagon; a hexagon; an octagon; a trapezoid; a parallelogram; a rectangle; or other geometric shape, including any of the shapes disclosed in U.S. patent application Ser. No. 17/157,322, incorporated by reference herein. In an embodiment, each of the plurality of top lattice apertures 220 may be configured as a diamond shape.

In an embodiment, the articulating interbody fusion device 1000 may have a plurality of front lattice apertures 240. The plurality of front lattice apertures 240 may be arranged in a two-dimensional array throughout the front surface. In an embodiment, the two-dimensional array of front lattice apertures 240 may be configured so the adjacent rows and/or columns may be staggered. Alternatively, the two-dimensional array of front lattice apertures 240 may be configured so the adjacent rows and/or columns may be equally spaced. The plurality of front lattice apertures 240 may extend through the body 100 of the articulating interbody fusion device 1000 providing continuous pathways from the front side of the porous structure to the opposing rear side of the porous structure. In an embodiment, each of the plurality of front lattice apertures 240 may be configured with any of the shapes mentioned above. In an embodiment, each of the plurality of front lattice apertures 240 may be configured as an oval shape.

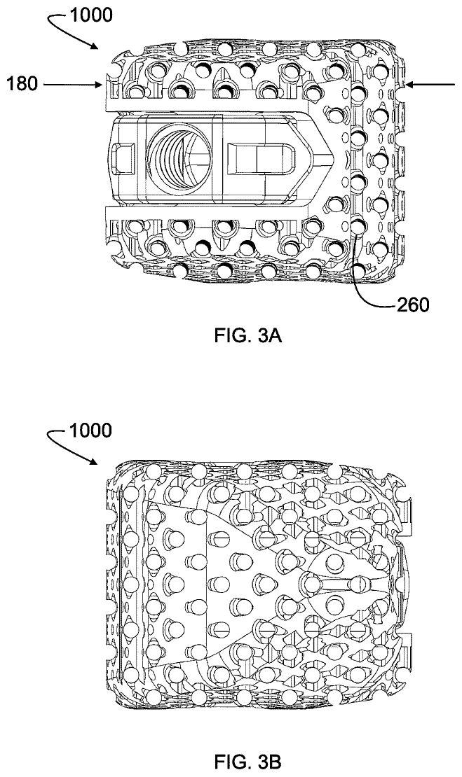

FIG. 3A is a right-side view of the articulating interbody fusion device 1000. FIG. 3B is a left-side view of the articulating interbody fusion device 1000. A side view may represent a medial-lateral plane, i.e., a view along the medial-lateral direction, orthogonal to a plane perpendicular to the medial-lateral direction. In an embodiment, the articulating interbody fusion device 1000 may have a width 180. The width 180 may represent a distance from a front end to a back end as measured in a medial-lateral plane. The articulating interbody fusion device 1000 may be configured such that the width 180 is within a range of widths from 5 mm to 20 mm. In an embodiment, the articulating interbody fusion device 1000 may be one of a set of differently-sized implants, each having a different width 180.

In an embodiment, the articulating interbody fusion device 1000 may have a plurality of side lattice apertures 260. The plurality of side lattice apertures 260 may arranged in a two-dimensional array throughout the front surface. In an embodiment, the two-dimensional array of side lattice apertures 260 may be configured so the adjacent rows and/or columns may be staggered. Alternatively, the two-dimensional array of side lattice apertures 260 may be configured so the adjacent rows and/or columns may be equally spaced. The plurality of side lattice apertures 260 may extend through the body 100 of the articulating interbody fusion device 1000 providing continuous pathways from one side of the porous structure to an opposing side of the porous structure. In an embodiment, each of the plurality of side lattice apertures 260 may have any of the shapes set forth above. In an embodiment, each of the plurality of side lattice apertures 260 may be configured as a circular shape.

In an embodiment, the plurality of top lattice apertures 220, the plurality of front lattice apertures 240, and/or the plurality of side lattice apertures 260 may intersect within the body 100 of the articulating interbody fusion device 1000. The intersection may result in a porous structure with a plurality of interconnected pores. The irregular shape of the interconnected pores may facilitate bone in-growth through the articulating interbody fusion device 1000 in a non-linear manner. The non-linear bone in-growth may provide greater integration of the articulating interbody fusion device 1000 within a fused vertebral joint and may prevent expulsion, subsidence and/or migration of the articulating interbody fusion device 1000 from the intervertebral space. In an embodiment, the porous structure may have a modulus of elasticity similar to that of a bone.

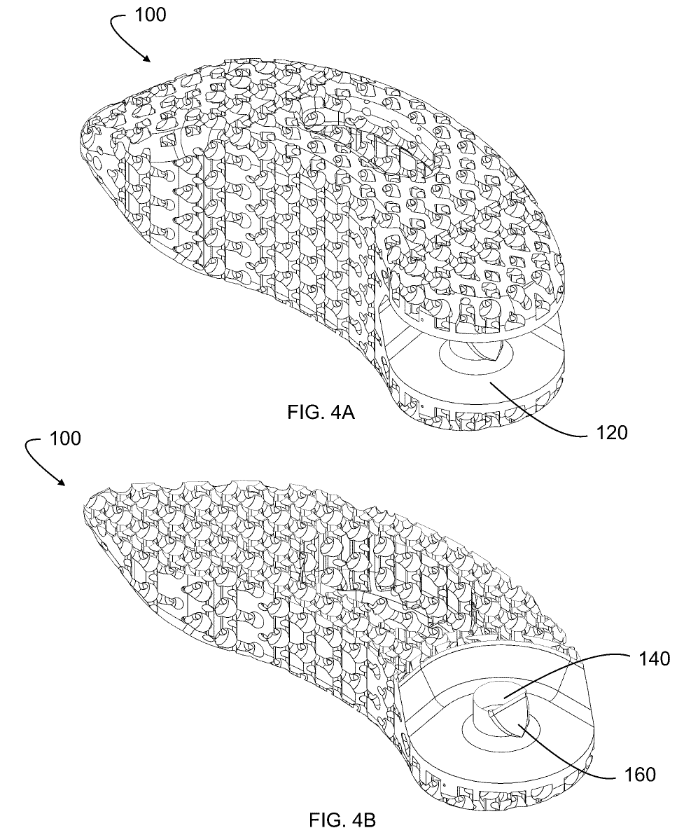

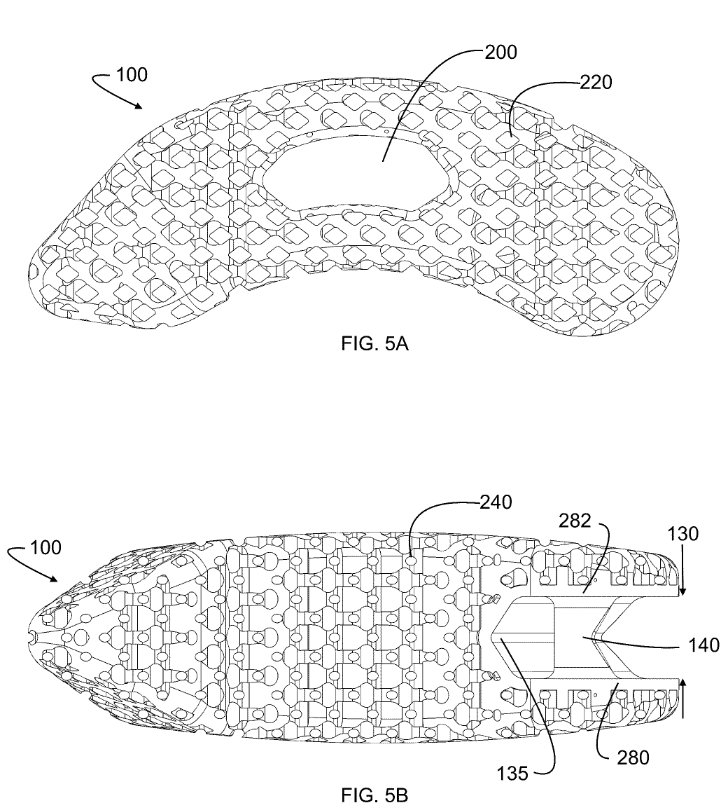

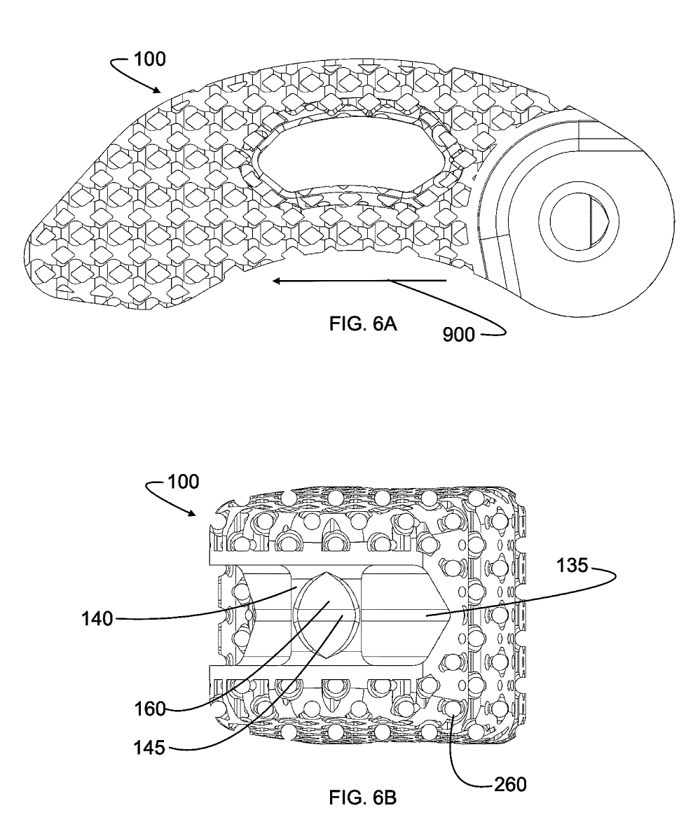

FIG. 4A is a perspective view of a body 100 of an articulating interbody fusion device 1000. FIG. 4B is a perspective section view of the body 100, FIG. 5A is a top view of the body 100, FIG. 5B is a front view of the body 100, FIG. 6A is a top section view of the body 100, and FIG. 6B is a right-side view of the body 100.

The articulating interbody fusion device 1000 may have an articulating connector 500 movably coupled to a post 140 within a recess portion 120 of the body 100. The recess portion 120 may have a recess height 130 and a post 140. The recess height 130 may be greater than or generally equal to the connector height 590 to allow rotation of the articulating connector 500 about the post 140. The post 140 may have a locking feature 160 configured to detachably connect to a distal portion of an inserter device (not shown). The post 140 may extend along the entire recess height 130. The post 140 may be monolithically formed with the body 100. The articulating connector 500 may be formed encircling the post 140 so that the articulating connector 500 may be irremovable from the body 100.

In an embodiment, the recess portion 120 may be configured with a first border portion 280 and a second border portion 282 spaced apart from the first border portion 280, each lacking a top lattice aperture 220; a front lattice aperture 240; and a side lattice aperture 260. The portion of the body 100 between the second border portion 282 and the top surface of the body 100 and the portion of the body 100 between the first border portion 280 and the bottom surface of the body 100 may each have a plurality of top lattice apertures 220. The post 140 may extend from the first border portion 280 to the second border portion 282.

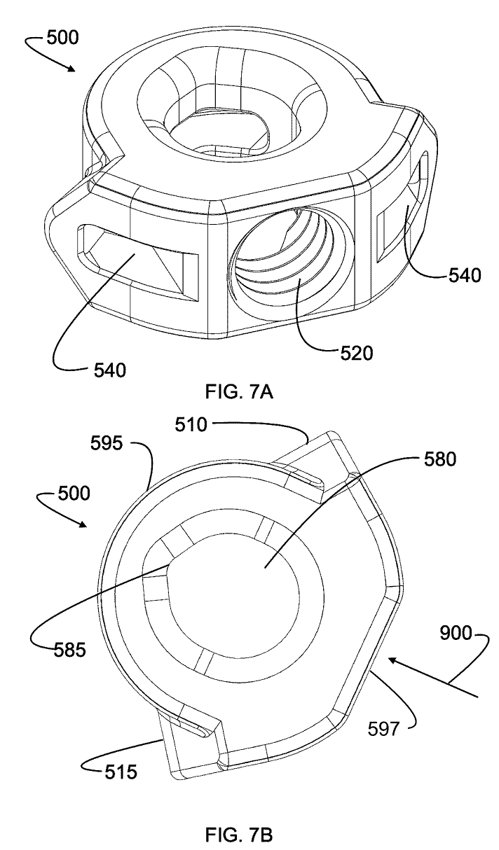

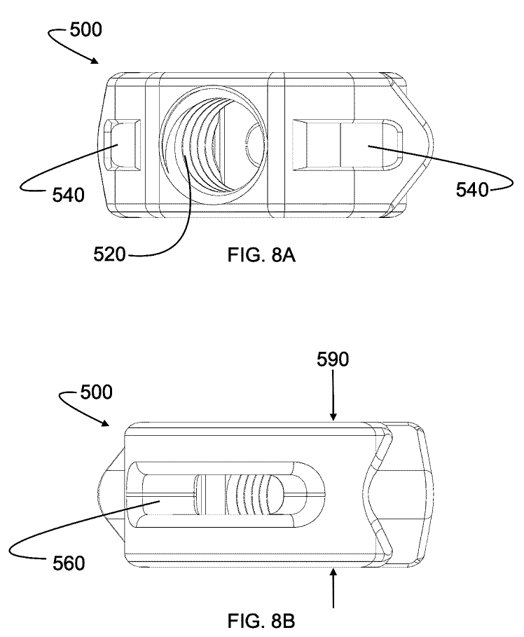

FIG. 7A is a perspective view of an articulating connector 500 of an articulating interbody fusion device 1000 according to an embodiment of the present disclosure. FIG. 7B is a top view of the articulating connector 500, FIG. 8A is a right-side view of the articulating connector 500, and FIG. 8B is a left-side view of the articulating connector 500. The articulating connector 500 may be configured to rotatably engage a post 140 of a body 100 within a recess portion 120 of the body 100. The articulating connector 500 may be configured to detachably connect to an inserter device (not show). The articulating connector 500 may have a connection feature 520 and one or more connection notches 540. In an embodiment, the connection feature 520 may be configured as a female thread extending from a proximal surface 597 and the inserter device may have a corresponding threaded distal portion. The connection feature 520 may be configured to allow the implant insert to advance through the connection feature 520 and into an articulating aperture 580.

The articulating connector 500 may include an outer perimeter 595 defining a continuous line forming the outside boundary of the articulating connector 500. The articulating aperture 580 may be entirely encircled by the outer perimeter 595. The articulating connector 500 may be formed as a single monolithic piece so that, with the post 140 received within the articulating aperture 580, the articulating connector 500 is irremovable from the body 100.

The articulating aperture 580 may be configured to be rotatable relative to a post 140 of a body 100. The post 140 may be positioned within the articulating aperture 580. The one or more connection notches 540 may be configured to engage a portion of the inserter device (not shown) and may be configured to facilitate deployment of the articulating interbody fusion device 1000 into an intervertebral space. Additionally, or alternatively, the one or more connection notches 540 may be configured to secure the connection between the articulating interbody fusion device 1000 and the inserter device (not shown) in a manner that prevents relative rotation between the articulating connector 500 and the inserter. In an embodiment, the articulating connector 500 may have a connector height 590 configured to be less than or equal to a recess height 130 of a body 100. The articulating connector may further have a rear aperture 560.

The articulating connector 500 may have a first stop surface 510 and a second stop surface 515 configured to limit the rotational movement of the articulating connector 500 within the recess portion 120. In an embodiment, the first stop surface 510 may be configured to limit rotational movement of the articulating connector 500 at a first position and the second stop surface 515 may be configured to limit rotational movement of the articulating connector 500 at a second position, wherein the angle between the first position and the second position is a rotation angle 820. In an embodiment, the rotation angle 820 may be in a range of 30° to 90°. More specifically, the rotation angle 820 may be in a range of 50° to 70°. More specifically, the rotation angle 820 may be 60°.

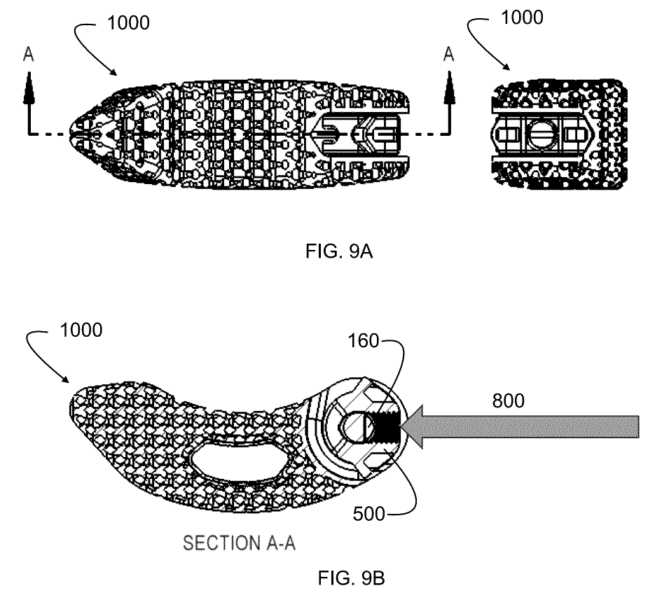

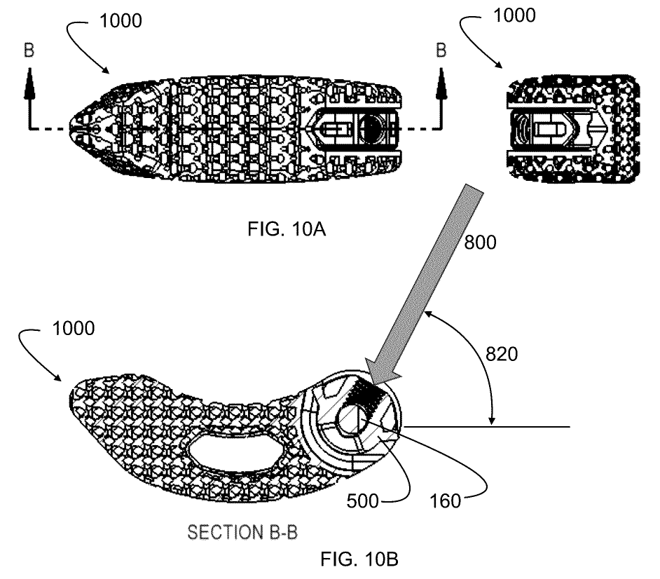

FIG. 9A is a front view and a right-side view of an articulating interbody fusion device 1000 with the articulating connector 500 in an insertion position according to an embodiment of the present disclosure. FIG. 9B is a top section view of the articulating interbody fusion device 1000. FIG. 10A is a front view and a right-side view of the articulating interbody fusion device 1000 with the articulating connector 500 in an implanted position according to an embodiment of the present disclosure. FIG. 10B is a top section view of the articulating interbody fusion device 1000. In an embodiment, the articulating interbody fusion device 1000 may be configured to detachably connect to an inserter device (not shown). The inserter device may detachably connect to the articulating interbody fusion device 1000 at an insertion orientation 800.

The articulating interbody fusion device 1000 may have an insertion position and an implanted position. In the insertion position, the inserter device may detachably connect to the connection feature 520 of the articulating connector 500 and also engage the locking feature 160 of the post 140 of the body 100 wherein engagement of the locking feature 160, through the connection feature 520, may prevent rotation of the articulating connector 500 about the post 140 of the body 100. In the implanted position, the inserter device may be partially disengaged from the connection feature 520 wherein the inserter device may not engage the locking feature 160 but remains engaged with the connection feature 520. In the implanted position, the inserter device may facilitate rotation of the articulating connector 500 about the post 140 along a rotation angle 820.

Any of the devices described herein may be manufactured from metals, alloys, polymers, plastics, ceramics, glasses, composite materials, or combinations thereof, including but not limited to: titanium, titanium alloy, stainless steel, PEEK (polyether ether ketone), among others. Different materials may be used within a single part.

Any of the devices described herein may be manufactured utilizing an additive manufacturing process. Additive manufacturing processes may include, but are not limited to, three-dimensional printing (3DP) processes, laser-net-shape manufacturing, direct metal laser sintering (DMLS), direct metal laser melting (DMLM), plasma transferred arc, freeform fabrication, direct digital manufacturing, layered manufacturing, and rapid prototyping. Additive manufacturing may be performed utilizing a process in which material is deposited, joined, or solidified, with the material being added together layer by layer.

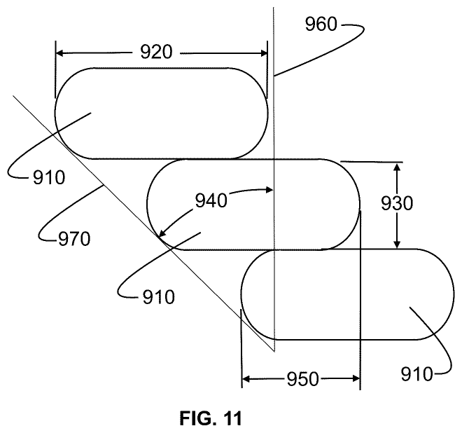

FIG. 11 is a schematic representation of additive manufacturing material layers 910. Each additive manufacturing material layer 910 may have a path width 920 and a layer height 930. The path width 920 and the layer height 930 may be dependent on a specific additive manufacturing process, a specific equipment manufacturer, and/or process parameters.

An additive manufacturing process may also have a percentage overlap 950 wherein a subsequent layer overlaps a previous layer in order to create a sloped surface. The path width 920, the layer height 930, and the percentage overlap 950 may be used to calculate a maximum overhang angle 940. The maximum overhang angle 940 may define an angle between a tangent edge 970 of a plurality path widths 920 and a vertical printing axis 960 aligned with the printing orientation 900. All additively manufactured overhang surfaces with an angle, as measured from the vertical printing axis 960, greater than the maximum overhang angle 940 may require support structures as part of the additive manufacturing process.

The maximum overhang angle 940 may be calculated using the following formula:

$$a = {tan}^{- 1} \frac{d * ( 1 - f )}{h}$$

where:
a=maximum overhang angle 940
d=path width 920
f=percentage overlap 950 (industry default is 33%)
h=layer height 930

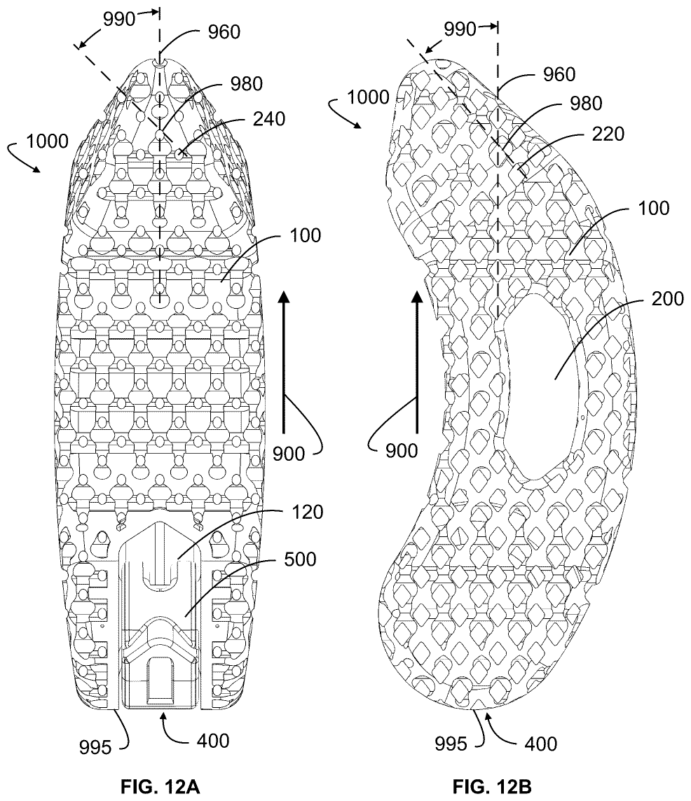

FIG. 12A is a front view of an articulating interbody fusion device 1000 according to an embodiment of the present disclosure. FIG. 12B is a top view of the articulating interbody fusion device 1000. The articulating interbody fusion device 1000 may be oriented for additive manufacturing such that a proximal end 400 includes a build plate contact surface 995. The articulating interbody fusion device 1000 may be aligned along a printing orientation 900 such that the printing orientation 900 is generally parallel to a vertical printing axis 960. The additive manufacturing process may form the articulating interbody fusion device 1000 starting at the build plate contact surface 995 and proceeding along the vertical printing axis 960.

Each of the plurality of front lattice apertures 240 and the plurality of top lattice apertures 220 may include an overhang surface 980 and an overhang angle 990. Additionally, or alternatively, an overhang surface 980 may be any surface, within the articulating interbody fusion device 1000, directly adjacent to a portion devoid of material, wherein the portion devoid of material is positioned between the overhang surface 980 and the build plate contact surface 995. The recess portion 120 may include an overhang surface 980. The central aperture 200 may include an overhang surface 980.

The overhang angle 990 may be an angle measured between the vertical printing axis 960 and a line parallel and/or tangent to an overhang surface 980. The overhang angle 990 may be one of a plurality of overhang angles 990 resulting from the geometry, position, and/or orientation of the overhang surface 980.

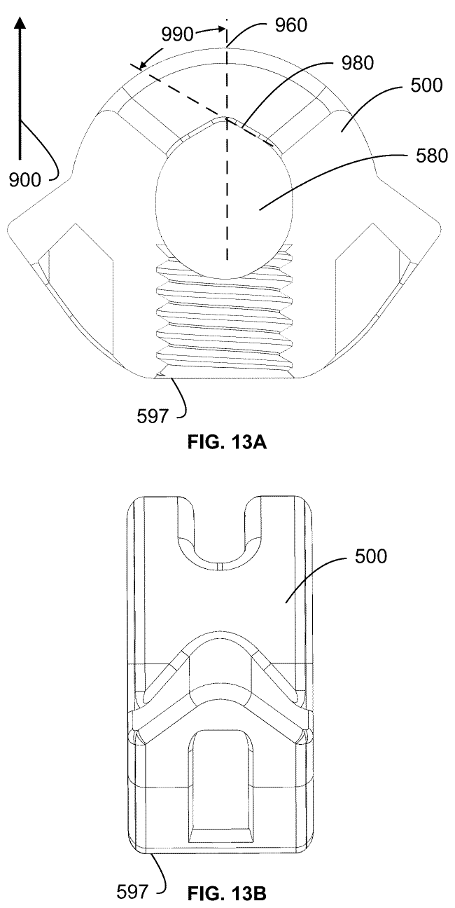

FIG. 13A is a top section view of an articulating connector 500 according to an embodiment of the present disclosure. FIG. 13B is a side view of the articulating connector 500. The articulating connector 500 may be oriented within the recess portion 120 of the body 100 for additive manufacturing such that a proximal surface 597 is generally parallel to a build plate contact surface 995. In an embodiment, the proximal surface 597 may be coplanar with the build plate contact surface 995. The articulating connector 500 and the body 100 may be additively manufactured as an assembly such that the articulating connector 500 is irremovable from the body 100.

The articulating connector 500 may be aligned along a printing orientation 900 such that the printing orientation 900 is generally parallel to a vertical printing axis 960. The additive manufacturing process may form the articulating connector 500 and the body 100 starting at the build plate contact surface 995, and/or the proximal surface 597, and proceeding along the vertical printing axis 960.

The articulation aperture 580 may include an overhang surface 980 and an overhang angle 990. Additionally, or alternatively, an overhang surface 980 may be any surface, within the articulating connector 500, directly adjacent to a portion devoid of material, wherein the portion devoid of material is positioned between the overhang surface 980 and the proximal surface 597. The connection notch 540 may include an overhang surface 980.

The articulating interbody fusion device 1000 may include one or more angled overhang surfaces 980 that are not perpendicular to a printing orientation 900 so that the articulating interbody fusion device 1000 may be additively manufactured as an assembly without support structures for the angled overhang surfaces 980.

The articulating interbody fusion device 1000 may be configured so that all overhang surfaces 980 are configured with an overhang angle 990, as measured from the vertical printing axis 960, less than the maximum overhang angle 940. The articulating interbody fusion device 1000 may be one of a set of differently configured implants, wherein each has a plurality of interconnected pores configured so that all overhang surfaces 980 are configured with an overhang angle 990, as measured from the vertical printing axis 960, less than the maximum overhang angle 940 for a given additive manufacturing process. The interconnected pores may provide continuous pathways from a first side of the body 100 to an opposing second side of the body 100.

In an embodiment, the articulating interbody fusion device 1000 may have a body 100 and an articulating connector 500 wherein both the body 100 and the articulating connector 500 may be manufactured in a single additive manufacturing process in an assembled configuration.

The articulating interbody fusion device 1000 may have a printing orientation 900 wherein printing begins at the proximal end 400 and advances in the direction of the distal end 300. The articulating interbody fusion device 1000 may be oriented during the additive manufacturing process so that the distal end 300 is facing upwards during the additive manufacturing process. The additive manufacturing process may begin at the proximal end 400 and continue layer by layer ending at the distal end 300.

The body 100 may have a porous structure configured so that the pores are oriented in a manner such that the internal porosity builds without support material during the additive manufacturing process.

Each of the plurality of top lattice apertures 220 may be configured as a geometric shape with a height along the printing orientation 900 and a width perpendicular to the printing orientation 900, wherein the height along the printing orientation 900 is greater than the width perpendicular to the printing orientation 900. Additionally, each of the plurality of front lattice apertures 240 may be configured as a geometric shape with a height along the printing orientation 900 and a width perpendicular to the printing orientation 900, wherein the height along the printing orientation 900 is greater than the width perpendicular to the printing orientation 900.

The body 100 and the articulating connector 500 may be printed in an assembled configuration without support material or struts connecting the body 100 to the articulating connector 500. The body 100 may be additively manufactured as a porous structure. The articulating connector 500 may have porosity resulting from the additive manufacturing process. Additionally, or alternatively, the articulating connector 500 may have a porous structure composed of a plurality of interconnected pores.

The articulating interbody fusion device 1000 may be configured so that, when the articulating interbody fusion device 1000 is oriented with the distal end 300 upward during an additive manufacturing process, an angle measured between an overhang surface 980 within the articulating interbody fusion device 1000 and an additive manufacturing build plate is greater than 30°.

The recess portion 120 of the body 100 may have a recess notch 135 and a post notch 145 each with an included angle between 70° and 120° and configured such that, when additively manufactured in the printing orientation 900, the recess portion 120, the recess notch 135 and the body 100 may be printed without the use of support material.

The articulating connector 500 may have an aperture notch 585 with an included angle between 70° and 120° and configured such that, when additively manufactured in the printing orientation 900, the articulating connector 500 may be printed without the use of support material. The aperture notch 585 may be located on a distal surface of the articulating connector opposite the connection feature 520.

The articulating interbody fusion device 1000, more specifically the body 100 and the articulating connector 500, more specifically the recess notch 135, the post notch 145, the connection notch 540 and the aperture notch 585 may have angled overhang surfaces that are not perpendicular to the printing orientation 900 so that the articulating interbody fusion device 1000 may be additively manufactured as an assembly without support structures. Additionally, the articulating interbody fusion device 1000 may be additively manufactured so that the body 100 is a porous structured and the articulating connector 500 is positioned to be rotatably coupled to the porous structure. Additionally, additively manufacturing the body 100 and additively manufacturing the articulating connector 500 may include additively manufacturing the body 100 and the articulating connector 500 together, with the body 100 and the articulating connector 500 uncoupled to each other by support material, aside from a junction between the post and the remainder of a support structure.

The body 100 may be printed as a porous structure wherein the body 100 includes a post 140 within the articulation aperture 580 of the articulating connector 500. Additionally, additively manufacturing the body 100 may also include additively manufacturing the body 100 with the post 140 and a remainder of the body 100 uncoupled to each other by support material, aside from a juncture between the post 140 and the remainder of the support structure.

In an embodiment, the articulating connector 500 may be connected to the body 100 only through removeable support material utilized within the additive manufacturing process. Additionally, or alternatively, break-away struts may be added to connect the articulating connector 500 to the body 100 during the additive manufacturing process whereby the struts may be broken and/or removed after completion of the additive manufacturing process to allow the articulating connector 500 to rotate within the body 100.

The method for deploying an articulating interbody fusion device 1000 using an inserter device according to an embodiment may include the following steps:
1. Facetectomy and disc space preparation: depending on the pathology, it may be necessary to partially or fully resect the facet joint and/or the lamina. A facetectomy and/or laminotomy may be performed using the surgeon's preferred technique and instruments.
2. Distraction: gradually distract the disc space using the paddle distractors. The final disc height achieved should be consistent with the disc heights of the adjacent levels.
3. Discectomy and endplate preparation: use shavers to continue the discectomy and prepare the vertebral endplates.
4. Implant size selection: select implant size (height, length, and lordosis) best suited to the patient's anatomy.
5. Articulating interbody fusion device preparation: The central aperture may be filled with allograft or autograft.
6. Inserter: detachably connect the inserter device to the articulating interbody fusion device, ensure the inserter device is fully seated within the articulating interbody fusion device.
7. Articulating interbody fusion device insertion: insert the articulating interbody fusion device into the interbody space with successive impactions on the implant inserter.
8. Final positioning: loosen the inserter device from the articulating interbody fusion device to allow rotation of the articulating connector. Use inserter device to pivot the articulating interbody fusion device into the final position.
9. Inserter device removal: detach the inserter device from the articulating interbody fusion device and remove.

Those of skill in the art will recognize that this is only one of many potential methods that may be used for deploying an articulating interbody fusion device. Further, the method may be employed to deploy an articulating interbody fusion device using an inserter device besides that which is specifically disclosed herein.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

The phrases “generally parallel” and “generally perpendicular” refer to structures that are within 30° parallelism or perpendicularity relative to each other, respectively. Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles of the disclosure.

While specific embodiments and applications of the present disclosure have been illustrated and described, it is to be understood that the disclosure is not limited to the precise configuration and components disclosed herein. Various modifications, changes, and variations which will be apparent to those skilled in the art may be made in the arrangement, operation, and details of the methods and systems of the present disclosure without departing from its spirit and scope.

What is claimed is:

1. An articulating interbody fusion device comprising:
a body comprising:
a plurality of interconnected pores; and
a recess portion comprising a first border portion and a second border portion spaced apart from the first border portion; and
an articulating connector configured to be positioned within the recess portion;
wherein:
one of the body and the articulating connector comprises an aperture;
the other of the body and the articulating connector comprises a post positioned within the aperture;
the articulating connector is rotatable relative to the body; and
the articulating connector is irremovable from the body.

2. The articulating interbody fusion device of claim 1, wherein the post is monolithically formed with the body.

3. The articulating interbody fusion device of claim 2, wherein the post extends from the first border portion to the second border portion.

4. The articulating interbody fusion device of claim 1, wherein the articulating connector comprises the aperture and an outer perimeter, wherein the aperture is entirely encircled by the outer perimeter.

5. The articulating interbody fusion device of claim 1, wherein the first border portion and the second border portion lack interconnected pores.

6. The articulating interbody fusion device of claim 1, wherein the plurality of interconnected pores provides continuous pathways from a first side of the body to an opposing second side of the body.

7. The articulating interbody fusion device of claim 1, wherein the body comprises a modulus of elasticity similar to that of a bone.

8. The articulating interbody fusion device of claim 1, further comprising a printing orientation, wherein the articulating interbody fusion device comprises one or more overhang surfaces that are not perpendicular to the printing orientation.

9. The articulating interbody fusion device of claim 1, wherein the articulating connector is formed as a single monolithic piece.

10. The articulating interbody fusion device of claim 1, wherein the articulating connector further comprises:
a first stop surface configured to limit rotational movement of the articulating connector at a first position; and
a second stop surface configured to limit rotational movement of the articulating connector at a second position;
wherein an angle between the first position and the second position is in a range of 50° to 70°.

11. A method of manufacturing an articulating interbody fusion device, the method comprising:
additively manufacturing a body; and
additively manufacturing an articulating connector such that the articulating connector is positioned to rotatably engage the body;
wherein additively manufacturing the body and the articulating connector comprises additively manufacturing the body and the articulating connector together, with the body and the articulating connector uncoupled to each other by a support material during manufacturing.

12. The method of claim 11, wherein additively manufacturing the body comprises forming one or more notches on a distal surface of the body and additively manufacturing the body without utilizing the support material.

13. The method of claim 11, wherein additively manufacturing the articulating connector comprises forming a notch on the articulating connector and additively manufacturing the articulating connector without utilizing the support material.

14. The method of claim 11, wherein:
the body comprises a post positioned within an aperture of the articulating connector; and
additively manufacturing the body comprises additively manufacturing the body with the post and a remainder of the body uncoupled to each other by the support material, aside from a junction between the post and the remainder of a support structure.

15. The method of claim 14, wherein additively manufacturing the body comprises forming a notch on the post and manufacturing the post without utilizing the support material.

16. The method of claim 11, wherein additively manufacturing the body and additively manufacturing the articulating connector comprise forming one or more angled overhang surfaces that are not perpendicular to a printing orientation so that the articulating interbody fusion device is additively manufactured as an assembly without support structures.

17. The method of claim 11, wherein the articulating connector is additively manufactured such that the articulating connector is irremovable from the body.

18. The method of claim 11, wherein:
additively manufacturing the body comprises:
forming a plurality of interconnected pores;
forming a recess portion comprising a first border portion lacking the interconnected pores and a second border portion lacking the interconnected pores spaced apart from the first border portion; and
forming a post extending from the first border portion to the second border portion; and
additively manufacturing the articulating connector comprises forming the articulating connector within the recess portion, wherein the articulating connector comprises an outer perimeter and an aperture entirely encircled by the outer perimeter;
wherein:
the post is positioned within the aperture; and
the articulating connector is rotatable relative to the post.

19. The method of claim 11, wherein additively manufacturing the articulating connector further comprises:
forming a first stop surface configured to limit rotational movement of the articulating connector at a first position; and
forming a second stop surface configured to limit rotational movement of the articulating connector at a second position;
wherein an angle between the first position and the second position is in a range of 50° to 70°.

20. The method of claim 11, wherein additively manufacturing the body and the articulating connector comprises depositing layers so that all overhang surfaces are configured with an angle less than a maximum overhang angle.

21. An articulating interbody fusion device formed by:
additively manufacturing a body; and
additively manufacturing an articulating connector together with the body such that the articulating connector is positioned to rotatably engage the body;
wherein the articulating interbody fusion device comprises:
the body comprising:
a plurality of interconnected pores;
a recess portion comprising a first border portion lacking the interconnected pores and a second border portion lacking the interconnected pores spaced apart from the first border portion; and
a post extending from the first border portion to the second border portion; and
the articulating connector positioned within the recess portion;
wherein:
the articulating connector comprises an outer perimeter and an aperture entirely encircled by the outer perimeter;
the post is positioned within the aperture; and
the articulating connector is irremovable from the body.

22. The articulating interbody fusion device of claim 21, wherein the articulating interbody fusion device comprises one or more angled overhang surfaces that are not perpendicular to a printing orientation so that the articulating interbody fusion device is additively manufactured as an assembly without support structures for the angled overhang surfaces.

23. The articulating interbody fusion device of claim 21, wherein additively manufacturing the body and the articulating connector comprises additively manufacturing the body and the articulating connector uncoupled to each other by support material during manufacturing.

24. The articulating interbody fusion device of claim 21, wherein additively manufacturing the articulating connector comprises forming one or more notches on a distal surface of the articulating connector and additively manufacturing the articulating connector without utilizing a support material.

25. The articulating interbody fusion device of claim 21, wherein additively manufacturing the body and the articulating connector comprises depositing layers so that all overhang surfaces are configured with an angle less than a maximum overhang angle.
